(19) United States
(12) Patent Application Publication
(10) Pub. No.: US 2026/0102600 A1
(43) Pub. Date: Apr. 16, 2026

(54) Catheter Valve, Catheter and Assembly Method

(71) Applicant: PulseCath B.V., Breukelen (NL)

(72) Inventor: Oren Malchin, Maarssen (NL)

(21) Appl. No.: 19/116,649

(22) PCT Filed: Sep. 30, 2022
(86) PCT No.: PCT/NL2022/050550

§ 371 Date: Mar. 28, 2025

(51) Int. Cl. A61M 39/24; A61M 39/00

(52) U.S. Cl. CPC A61M 39/24; A61M 2039/0009; A61M 2039/244; A61M 2205/02; A61M 2205/3331; A61M 2207/10

(57) ABSTRACT

Catheter valve, including a valve housing defining a fluid channel, wherein the housing includes an asymmetric valve leaflet that is pivotal between a first position for allowing fluid flow through the fluid channel and a second position for closing the fluid channel, wherein the housing and valve leaflet are connected via a snap-fit connection. Further, there is provided a method for assembly of the catheter valve, including: providing the catheter housing; providing the valve leaflet; and snapping the valve leaflet into the housing.

### CROSS REFERENCE TO RELATED APPLICATION

The present application is a U.S. National Phase Application pursuant to 35 U.S. C § 371 of International Application No. PCT/NL2022/050550 filed on Sep. 30, 2022, the entire contents of which are herein incorporated by reference as if fully set forth in this description.

### BACKGROUND

The invention relates to a catheter valve.

WO2005/021078 discloses a catheter having a check valve, which can be used for closing a catheter channel. The valve can be located remote from a catheter inlet and catheter outlet. In the known configuration, the catheter is used for heart treatment, for example for controlling direction of blood flow. The document discloses a catheter extending through a ventricular valve of an active heart, wherein the check valve with a movable valve body is provided between the catheter's inlet passages and the outlet passage (in that case, to avoid creating a substantial open passage across a ventricular valve). According to the example, the valve body is pivotably suspended for rotation about an axis extending across the channel, between a closed position, substantially closing off the catheter channel and an open position allowing fluid to pass past the valve. In open position, the known valve body extends along a plane parallel to the channel and, in axial view, has a central portion spaced from the pivoting axis and, in side view, the valve body projects from the pivoting axis further in distal direction than in proximal direction. Starting from a closed position of the valve body, since (at least in open condition) the valve body projects from the pivoting axis further in distal direction than in proximal direction, blood pressure in distal direction results in the exertion of a couple on the valve body which causes it to pivot to its open position and it is held in that position by the blood flow in distal direction. After the stroke of a displacement structure urging blood in distal direction has ended, the displacement structure starts to draw blood in proximal direction. Already a small backflow at the valve body resulting from the reversal of the action of the displacement structure causes the valve body to pivot back to its closed position. Initial movement of the valve body is believed to be caused by a difference in flow velocities between the center and the periphery of the channel. As soon as the valve body is tilted a little in closing sense, the flow pushes against the surface of the valve body facing away from the axis, which causes the valve body to be pivoted into its closed position, blocking any substantial backflow.

Because, in open position, the known valve body extends along a plane parallel to the channel and, in axial view, has a central portion spaced from the pivoting axis, the valve leaves open a large portion of a lumen of the catheter for the passage of tools and causes very little resistance to the blood flow. Furthermore, opening and closing of the known valve does not require any material to bend, which is advantageous for obtaining reliable operation over prolonged periods of time.

For closing off the channel, which has a round cross-section, it is advantageous that the pivoting axis extends across a widest portion of the channel (measured in the direction of the axis), because this allows the known valve body to be wide enough to close off the channel along the entire circumference of the channel, while still allowing pivotal movement about the axis between the closed and open positions. Moreover, it is made possible that a section of the channel containing the valve body has a constant cross-section so that flow resistance is minimized, while nevertheless, the valve body in closed condition substantially abuts the continuing interior wall surface of the channel along the entire circumference of the channel.

The known valve body is a curved plate. In open position, the valve body leaves open passages between the plate and the catheter wall on both sides of the plate, so that blood can flow past the plate on both sides, which is favorable for avoiding areas of little flow where the risk of thrombosis is increased. The space between the plate and the interior wall of the channel when the plate is in open position also allows the plate to pivot to its closed position.

An example of a catheter-valve assembly is PulseCath's iVaC2L (see https://www.pulsecath.com/products/ivac-21/which is intended for use in patients with impaired left ventricular function which require left ventricular mechanical circulatory support for up to 24 hr. The iVAC 2L is a short term, fully percutaneous, 17Fr transfemoral LVAD that effectively generates blood flow up to 2 liters per minute. By actively unloading the ventricle, the iVAC 2L provides critical hemodynamic support during high-risk revascularization procedures, in cases of acute myocardial infarction and cardiogenic shock and for high-risk patients. The iVAC 2L incorporates a rotating 2-way valve which is connected to an extra corporeal membrane pump via a 17Fr. single lumen, 100 cm long catheter. It can be used with any standard IABP console and does not require dedicated hardware.

When the heart is in the systolic phase, blood is aspirated from the left ventricle through the catheter tip and lumen into the membrane pump.

During the diastolic phase the membrane pump ejects the blood back through the catheter, subsequently opening the catheter valve and delivering the blood to the ascending aorta through the side outflow port, thereby creating an “extra beat of the heart”. The pulsatile synchronization between the closing of the aortic valve and the opening of the catheter valve, ensures that aortic valve function is not impaired. The iVAC 2L directly unloads the heart by active aspiration from the left ventricle, and simultaneously creates a counter pulsating flow in the ascending aorta.

The known valves have opposite pivot axes that are part of a respective valve housing, the axes in particularly being welded to the housing.

These known valves performs relatively good, however, it has been found that the valve operation (i.e. movement of a valve leaflet between an open and closed position) can be relatively slow, which can lead to (backflow) leakage that reduces an outlet volume flow. Besides, manufacture of the known valve is relatively complex. In particular, manufacture involves providing two holes in the valve leaflet and two holes in the housing first, after which axels are inserted from both sides to create a pivot axial coupling. The axels welded are then welded to the housing body from outside to fix the axels in place.

### SUMMARY

The present invention aims to provide an improved catheter valve. In particular, the invention aims to provide a valve that has low leakage during operation and improved catheter outflow. Also, an aim is to improve valve manufacturability. Further, an aim is to provide a durable and reliable catheter valve.

According to the invention one or more of these goals can be achieved by the features of the independent claims.

In particular there is provided a catheter valve, including a valve housing defining a fluid channel, wherein the housing includes an asymmetric valve leaflet that is pivotal between a first position for allowing fluid flow through the fluid channel and a second position for closing the channel, wherein the housing and valve leaflet are connected via a snap-fit connection.

It has been found that in this way, improved valve manufacturing can be achieved. In particular, according to an embodiment, the present valve does not require to weld dedicated pivot axes into aligned positions to a valve housing. To the contrary, the housing can be provided with two opposite bores, for receiving valve pivot sections, wherein accurate alignment of the two bores can be achieved in a straightforward manner during manufacture (e.g. by maintaining the housing in a fixed position with respect to a boring tool, e.g. a CNC drilling device, when boring both pivot bores).

According to a preferred embodiment, the snap-fit connection (integrally) includes pivot joint sections (i.e. axles, protrusions) of the leaflet and the housing.

According to a further embodiment, the leaflet can be manufactured in one-piece, i.e. respective pivot joint sections of the leaflet can be made in once-piece with a remaining (central) section of the leaflet. For example, a suitable leaflet manufacturing process can be based on a wire cutting technique (also known as wire electrical discharge machining, see e.g. https://en.wikipedia.org/wiki/Electrial discharge machining). The resulting (‘snapon’) leaflet can be assembled to the valve housing as a spring without the need of welding (wherein assembly can involve spring biasing, e.g. elastically compressing, the leaflet). After assembly, the leaflet can be in a spring-relaxed state.

It has been found that this innovative improvement provides significant reduction of axials friction and can create a relatively smooth pivot movement of the leaflet. It can also increases leaflet speed (between different pivot positions with respect to the housing) whereas the outcome provides a more efficient valve and lower backflow leakage compared to the prior art valve assembly. Besides, it has been estimated that embodiments of the invention can reduce valve assembly time by 80%, by eliminating additional production stages such as welding. Also, this can decrease risks of malfunctioning due to welding heating that can effect housing dimensions.

In particular, it has been found that the resulting valve can achieve relatively swift valve leaflet repositioning, for example during blood flow reversal. In this way, significant reduction of leakage can be achieved. Valve prototype testing has achieved a leakage of less than 1%, compared to a 4-10% leakage of a prior art valve, which can lead to a significant increase of 4 cc (cubic centimeter) of blood per stroke volume (from 22 cc to 26 cc) during valve operation.

Besides, the present valve can provide reliable operation, and can provide a durable coupling between the valve leaflet and the housing.

Further, there is provided a catheter, comprising a catheter wall bounding a fluid channel (lumen), wherein a valve according to the invention is integrated in the catheter wall.

Besides, an aspect provides a method for assembly of a catheter valve according to the invention the method, including:
providing the catheter housing;
providing the valve leaflet; and
snapping the valve leaflet into the housing.

In this way, above-mentioned advantages can be achieved.

Further extra advantageous embodiments of the invention are provided in the dependent claims.

### BRIEF DESCRIPTION OF THE FIGURES

The invention will now be explained in more detail, with reference to the drawing. Therein shows:

FIG. 1 a top view of an embodiment of the invention, with the valve leaflet being in a first position;

FIG. 2 a side view of the embodiment;

FIG. 3 a cross-section over line III-III of FIG. 1;

FIG. 4 a cross-section over line IV-IV of FIG. 1;

FIG. 5 a similar cross-section as FIG. 4, showing the valve leaflet in a second position;

FIG. 6 an isometric view of the housing of the embodiment of FIG. 1;

FIG. 7 a top view of the housing;

FIG. 8 a cross-section over line VIII-VIII of FIG. 7;

FIG. 9 a cross-section over line IX-IX of FIG. 7;

FIG. 10 a top view of the leaflet of the embodiment of FIG. 1;

FIG. 11 a front view of the leaflet;

FIG. 12 a side view of the leaflet; and

FIG. 13 schematically part of a caterer including an embodiment of the valve.

Similar or corresponding features are denoted by similar or corresponding reference signs in this application.

### DETAILED DESCRIPTION

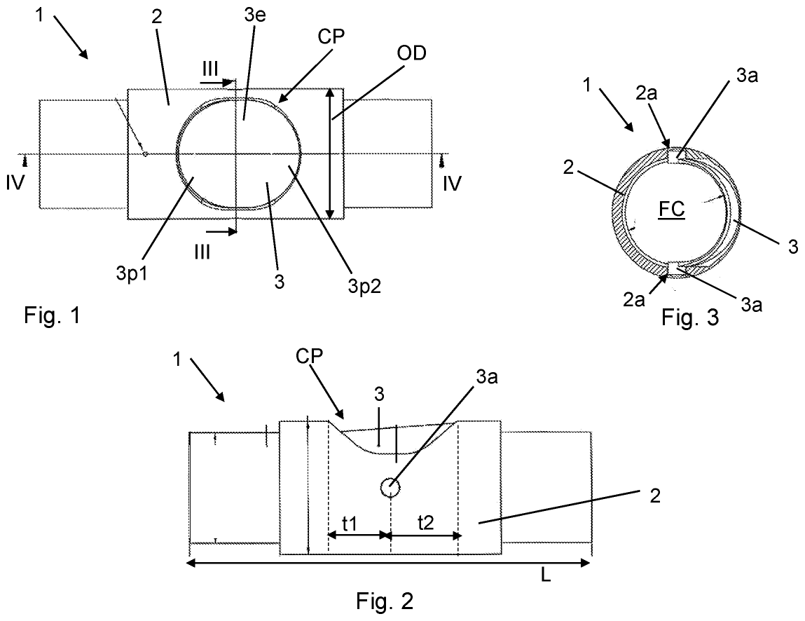

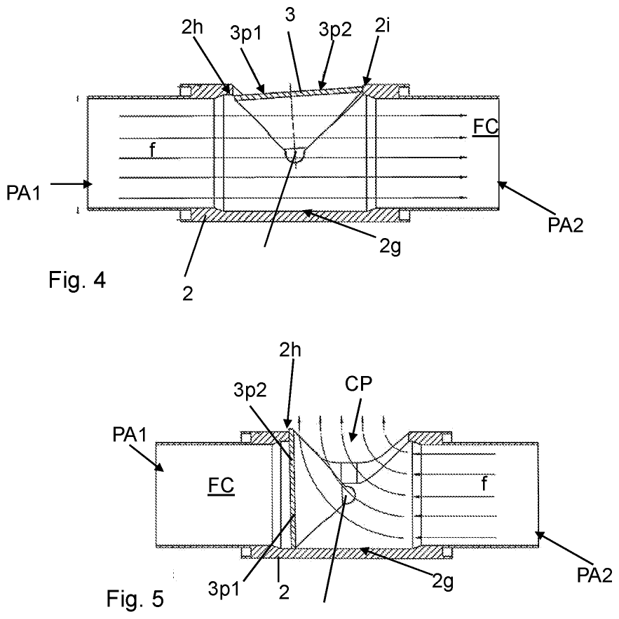

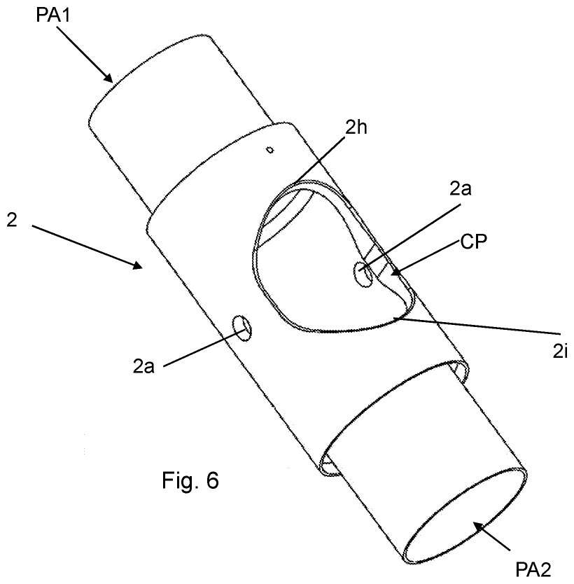

FIGS. 1-12 show a catheter valve 1, including a valve housing 2 defining a fluid channel FC. The housing 2 includes an asymmetric valve leaflet 3 that is pivotal between a first position (see FIG. 4) for allowing fluid flow through the fluid channel FC, and a second position (see FIG. 5) for closing the channel. Advantageously, the housing 2 and valve leaflet 3 are connected via a snap-fit connection 2a, 3a. The housing 2 can include a cross-flow port CP, wherein the valve leaflet 3 is arranged such that the leaflet closes the cross-flow port CP when the leaflet is in its first position and opens the cross-flow port CP when it is in its second position. The housing is shown in more detail in FIGS. 6-9.

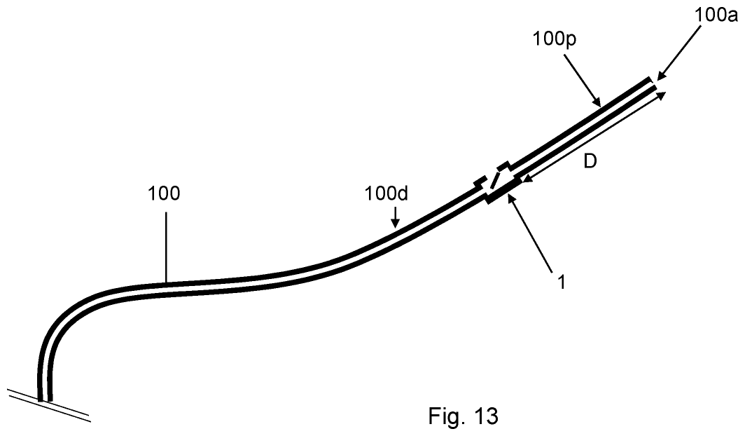

For example, the housing 2 can include a circle-cylindrical wall 2b that defines the (circle-cylindrical) flow channel FC. The wall 2b can e.g. be made of stainless steel. The cylindrical wall 2b can have integral cylindrical sleeve sections for receiving (and connecting) to catheter sections 100p, 100d, i.e. to be integrated in a catheter 100. An example of a resulting catheter is shown in FIG. 13. A maximum outer diameter OD of the cylindrical wall 2b can e.g. be 6 mm, whereas an inner diameter can e.g. be 1 mm smaller than the outer diameter. An outer diameter of the end sections 2c can be smaller than said maximum outer diameter OD, for fitting (snugging receiving) catheter ends of the catheter sections 100p, 100d thereon during catheter assembly. An overall axial length L of the housing 2 can be smaller than 30 mm, for example smaller than 20 mm (e.g. 18 mm).

The cylindrical valve housing wall 2b has two opposite circular bores 2a. In this example, each of the bores 2a protrudes radially (i.e. perpendicularly) through the wall 2b, from an inner surface to an outer surface of the wall 2b. Also, the bores 2a are located centrally, having bore centers that are located on a center longitudinal plane XP of the housing 2.

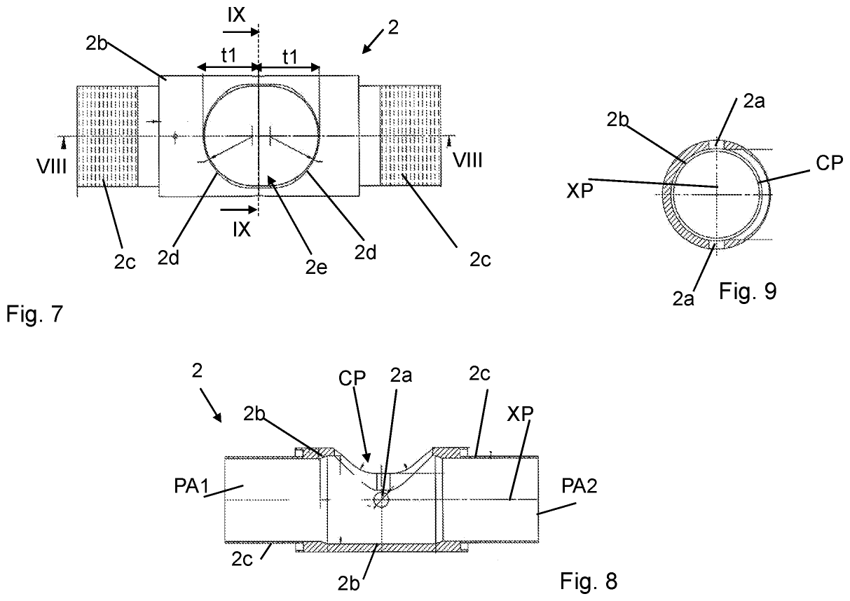

The outlet (cross-flow) port CP of the housing 2 can be an opening matching the shape of the valve leaflet 3. For example, the opening CP can be a saddle-shaped opening in the cylindrical wall 2b (i.e. an edge of the housing 2 defining the opening can be substantially saddle-shaped). In this example, the port CP can be defined by two semicircle sections 2d joined by an intermediate spacer-section 2e when viewed in a top view (see FIG. 7). The two bores 2a of the housing can be positioned (slightly) asymmetrically with respect to the cross-flow port CP, when viewed in the top view, i.e. an axial distance t1 between their pivot axis and a transversal plane intersecting a first longitudinal end of the port being smaller than an axial distance t2 between their pivot axis and a transversal plane intersecting an opposite second longitudinal end of the port, when viewed in top view or side view.

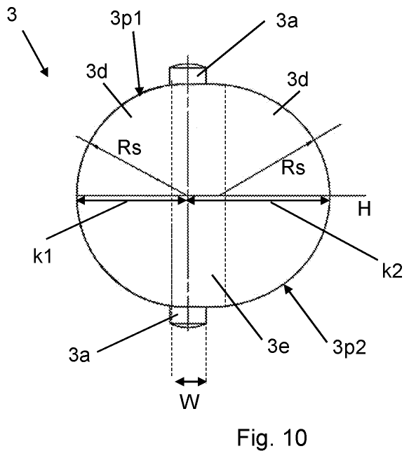

FIGS. 10-12 show the valve leaflet 3 in more detail. When viewed in a top view, the valve leaflet 3 can have two semicircle leaflet sections 3d joined by an intermediate spacer-section 3e that extends in parallel with the (virtual) pivot axis PA of the leaflet 3, the pivot axis PA being asymmetrically located with respect to the two semicircle sections. The leaflet's pivot axis PA can be defined by two integral, mutually aligned pivot axles 3a, in particular circle-cylindrical pivot joint sections 3a (the pivot axis PA extending centrally, concentrically, through these sections 3a). Therefore, the pivot axis can divide the leaflet 3 into a first part 3p1 and a second part 3p2 (see FIG. 12), the first part 3p1 being smaller (i.e. shorter) than the second part 3p2 (i.e. a length k1 of the first part 3p1 is smaller than a length k2 of the second part 3p2, both lengths k1, k2 being measured along a leaflet's virtual center plane H that extends normally with respect to the pivot axis PA).

The two integral pivot joint sections (axles) 3a of the leaflet 3 can e.g. protrude in opposite directions from a respective intermediate part 3e of the leaflet.

A diameter W of each of the pivot joint sections 3a can be slightly smaller than a diameter of the respective circle-cylindrical bores 2a of the valve housing 2, a diameter difference for example being at most 0.1 mm, preferably a diameter difference in the range of 0.04-0.08 mm. The diameter W of each of the leaflet's joint sections 3a can e.g. be smaller than 1 mm, for example a diameter W in the range of 0.7-0.9 mm.

The leaflet (which can also be called e.g. “platelet” or “valve element”) can consist of plate material having a thickness in the range of e.g. 100-500 microns, for example a thickness of about 0.2 mm. A radius Rs of each of the semicircle sections 3d (viewed in top view) can be the same, and can e.g. be in the range of 2.3-2.5 mm, for example about 2.4 mm.

According to a highly preferred embodiment, no welding is applied for providing the two (relatively small) two pivot joint sections 3a. In the that case, the two pivot joint sections (protrusions/axles) 3a can be made in one-piece with a remaining part of the leaflet 3, e.g. utilizing wire cutting.

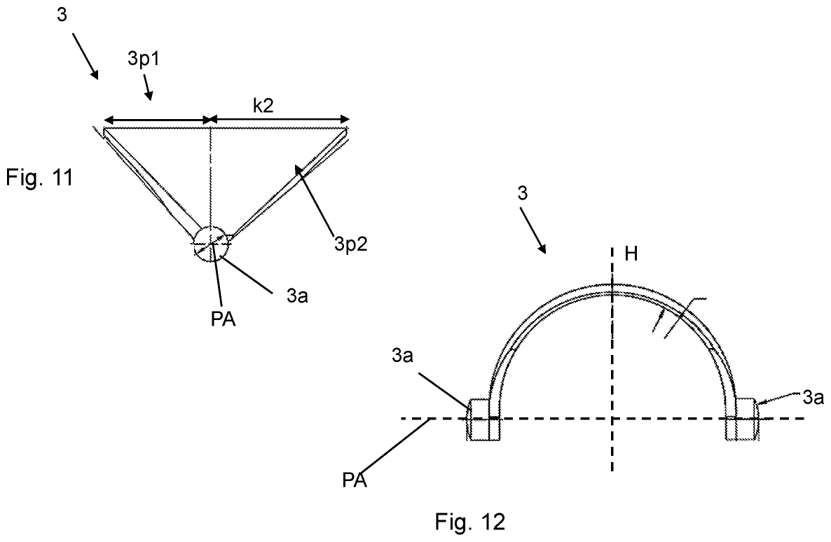

Also, —when viewed in a side view (i.e. in a direction that is normal to the leaflet's virtual pivot axis PA, see FIG. 12)—an inner surface of the valve leaflet 3 can extend along a virtual circular cylindrical plane. Besides, —when viewed in a front view (i.e. in a direction that is parallel to the pivot axis PA, see FIG. 11), the outer surface of the valve leaflet 3 can be substantially V-shaped.

If follows that the snap-fit connection can include the pivot joint sections 3a of the leaflet and the joint sections, in this case the circular aligned holes 2a, of the housing 2. In particular, the snap-fit connection can have cylindrical protrusions (axles) 3a of the leaflet 3 that are rotationally engaged by the opposite bores 2a of the housing 2.

The leaflet 3 can be spring-biased into place in the valve housing 2 (during assembly, when the leaflet is inserted into the housing). For example, assembly can involve pressing the two leaflet sides carrying the axles 3a towards each other (thereby elastically deforming the leaflet), against an internal spring force of the leaflet 3. Preferably, the leaflet is made of resilient (flexible, elastic) material, e.g. spring steel, stainless steel and/or Biodur® 108 stainless steel, to provide a respective spring force. Once the leaflet has been placed in the housing it is preferably in a spring-relaxed state (i.e. then, it does not achieve axial outwardly spring force, i.e. axially along the respective pivot axis PA).

During assembly (i.e. mounting the leaflet 3 into the housing), a leaflet generated spring force can be directed such that it counteracts inward movement of the two joint sections 3a of the leaflet 3. After assembly, the leaflet (as shown in FIG. 12) is preferably in a spring-relaxed (undeformed) state.

In particular, assembly of the catheter valve 1 can involve providing the catheter housing 2 (as shown in FIGS. 7-9) and providing the valve leaflet 3 (FIGS. 10-12). Next, the leaflet 3 can simply be snapped into place on the two bores 2a of the housing. The snapping can involve inwardly deforming (i.e. compressing) the leaflet 3, the leaflet counteracting leaflet deformation via integral spring force of leaflet spring material. As is mentioned before, preferably, once the leaflet 3 has been snapped into place (with the axles 3a being received in the respective openings 2a of the housing 2), the leaflet 3 can be in a spring relaxed (undeformed) state.

For example, the leaflet 3 can be introduced into the housing 2 via the side port CP or via one of the axial ports PA1, PA2 of the housing 2.

It is preferred that the leaflet 3 is positioned such that the longest leaflet section 3p2 is closest to the second side port PA2 and the shortest leaflet section 3p1 is located closest to the first side port PA1 of the housing, when the leaflet 3 is in its first position (engaging the housing 2).

It is preferred that the leaflet is positioned such that the longest leaflet section 3p2 is located away from a bottom 2g (see FIG. 5) of the housing 2 at any leaflet pivot position, whereas the shorter leaflet section 3p1 can move towards a bottom side 2g of the housing when the leaflet pivots from its first position to its second position (see FIG. 5 a well). As follows from the drawings, the bottom side 2g of the housing can be defined as an inner surface of the housing 2 that extends opposite to the outlet side port CP (the virtual pivot axis PA of the leaflet 3 extending between the side port CP and the bottom side 2g).

For example, the configuration can be such that after assembly, the (axial) edge of the shorter leaflet section 3p1 mechanically contacts the inner surface i.e. bottom side 2g of the housing 2 when the leaflet is in its second position (FIG. 5). Also, the configuration can be such that after assembly the (axial) edge of the longer leaflet section 3p2 mechanically contacts a first axial inner edge 2h of the side port CP when the leaflet 3 is in its second position (FIG. 5). For example, the latter first axial inner edge 2h can provide a first end stop to the longer leaflet section 3p2 for limiting further leaflet rotation (i.e. in a counterclockwise direction in FIG. 5).

Similarly, the configuration can be such that after assembly, the (axial) edge of the shorter leaflet section 3p1 mechanically contacts the first axial inner edge 2h of the side port CP when the leaflet 3 is in its first position (FIG. 4). Besides, the configuration can be such that after assembly the (axial) edge of the longer leaflet section 3p2 mechanically contacts an opposite second axial inner edge 2i of the side port CP when the leaflet 3 is in its first position (FIG. 4). For example, the latter second axial inner edge 2i can provide a second end stop (opposite to the first end stop) to the longer leaflet section 3p2 for limiting further leaflet rotation (i.e. in a clockwise direction in FIG. 4).

FIGS. 4 and 5 depict valve operation. As follows from FIG. 4, when the valve 1 is in a first state, with the leaflet 3 in its first position, fluid f (e.g. blood) can flow from the first axial end port PA1 to the second end port PA2 of the housing 2, whereas the leaflet 3 substantially closes the side port CP. The leaflet 3 can be held in this position due to fluid pressure of fluid f flowing from the first end port PA1 to the second end port PA2.

FIG. 5 shows the leaflet's position when fluid flow direction has been reversed. Due to pressure change on the leaflet 3, caused by the reversal of the flow direction, the asymmetric valve leaflet 3 has swiftly pivoted to its second position, thereby closing the channel in front of the first axial outlet port PA1 and opening the side port CP.

FIG. 13 schematically shows part a catheter 100 (in opened side view), being provided with the catheter valve 1. In particular, the catheter 100 can include catheter wall bounding a fluid channel (e.g. for conducting blood), wherein the valve 1 is integrated in the catheter wall, for example by the valve housing 2 being joined with (e.g. welded to) a proximal catheter section 100p and a distal catheter section 100d. The catheter 100 can e.g. be used in patients with impaired left ventricular function which require left ventricular mechanical circulatory, for example in the same way as the iVaC2L system (described above). For such an application it is preferred that the valve 1 is located at a distance D in the range of about 6-60 cm from a distal end 100a of the catheter.

While this disclosure includes specific example embodiments, it will be apparent to one of ordinary skill in the art that various changes in form and details may be made in these example embodiments without departing from the scope of the claims. The example embodiments described herein are to be considered in a descriptive sense only, and not for purposes of limitation.

1. A catheter valve, including:
a valve housing defining a fluid channel, wherein the valve housing includes an asymmetric valve leaflet that is pivotal between a first position for allowing fluid flow through the fluid channel and a second position for closing the fluid channel, wherein the valve housing and valve leaflet are connected via a snap-fit connection.

2. The catheter valve according to claim 1, wherein the valve leaflet is spring-biased into place in the valve housing during assembly, the valve leaflet in particular being made of resilient material to provide a respective spring force.

3. The catheter valve according to claim 1, wherein the snap-fit connection includes pivot joint sections of the valve leaflet and the valve housing.

4. The catheter valve according to claim 1, wherein the snap-fit connection is provided by integral cylindrical protrusions of the valve leaflet that are rotationally engaged by opposite bores of the valve housing.

5. The catheter valve according to claim 1, wherein the valve housing includes a cross-flow port, wherein the valve leaflet is arranged such that the valve leaflet closes the cross-flow port when the valve leaflet is in the first position and opens the cross-flow port it is in the second position.

6. The catheter valve according to claim 1, wherein—when viewed in a top view—the valve leaflet has two semicircle sections joined by an intermediate spacer-section that extends in parallel with a pivot axis of the valve leaflet, the pivot axis being asymmetrically located with respect to the two semicircle sections.

7. The catheter valve according to claim 5, wherein the cross-flow port is an opening matching a shape of the valve leaflet, the cross-flow port in particular being defined by two semicircle sections joined by an intermediate spacer-section when viewed in a top view.

8. The catheter valve according to claim 1, wherein—when viewed in a side view—an inner surface of the valve leaflet extends along a virtual circular cylindrical plane.

9. A catheter comprising:
a catheter wall bounding a fluid channel; and
a valve integrated in the catheter wall, wherein the valve comprises: a valve housing defining a portion of the fluid channel, wherein the valve housing includes an asymmetric valve leaflet that is pivotal between a first position for allowing fluid flow through the fluid channel and a second position for closing the fluid channel, wherein the valve housing and valve leaflet are connected via a snap-fit connection.

10. A method for assembly of a catheter valve, the method including:
providing a valve housing;
providing a valve leaflet; and
snapping the valve leaflet into the valve housing, against an internal spring force of the valve leaflet.

11. The method according to claim 10, further including:
boring two opposite bores in the valve housing, for receiving valve pivot sections of the valve leaflet.

12. The method according to claim 11, further including:
maintaining the valve housing in a fixed position with respect to a boring tool during the boring of the two opposite bores.

13. The method according claim 10, further including:
manufacturing the valve leaflet and respective pivot joint sections of the valve leaflet in one-piece from plate material utilizing wire electrical discharge machining.

14. A kit of parts including:
a catheter valve leaflet of a catheter valve the valve leaflet including integral pivot joint sections; and
a catheter valve housing of the catheter valve, the catheter valve housing including holes for pivotally receiving the integral pivot joint sections of the valve leaflet.

15. A method for manufacturing an asymmetric valve leaflet of a catheter valve, wherein the catheter valve includes a valve housing defining a fluid channel, wherein the asymmetric valve leaflet is pivotal between a first position for allowing fluid flow through the fluid channel and a second position for closing the fluid channel, wherein the valve housing and the asymmetric valve leaflet are connected via a snap-fit connection, wherein the snap-fit connection includes pivot joint sections of the asymmetric valve leaflet and the valve housing, the method including:
manufacturing the asymmetric valve leaflet and any respective pivot joint sections in one-piece from plate material.
